(19) United States
(12) Patent Application Publication
(10) Pub. No.: US 2023/0181787 A1
(43) Pub. Date: Jun. 15, 2023

(54) Ultraviolet Air Sterilizer

(71) Applicant: Henry K. Obermeyer, Wellington, CO (US)

(72) Inventor: Henry K. Obermeyer, Wellington, CO (US)

(21) Appl. No.: 17/926,456

(22) PCT Filed: May 21, 2021
(86) PCT No.: PCT/US2021/033773

§ 371 Date: Nov. 18, 2022

Related U.S. Application Data
(60) Provisional application No. 63/028,531, filed on May 21, 2020.

(51) Int. Cl. A61L 9/20

(52) U.S. Cl. CPC A61L 9/20; A61L 2209/12; A61L 2209/14

(57) ABSTRACT

The present invention discloses improved ultraviolet air sterilizers. In accordance with this invention the mean reflected light path of each photon is maximized while turbulence is suppressed in order to uniformly UVC expose all conveyed pathogen particles. This invention discloses novel means of maximizing the utilization of blower power and UVC lamp power toward the destruction of pathogens. The resulting efficiencies facilitate the construction of high flow rate air sterilization systems for large buildings while also facilitating the miniaturization of UVC air sterilization systems for incorporation into personal protective equipment (PPE).

### FIELD OF THE INVENTION

The present invention relates to sterilization of air by means of ultraviolet light, preferably in the ultraviolet-C(UVC) band (i.e., 200-280 nanometers wavelength). Many air purifying systems have been built that incorporate UVC light for germicidal purpose. The prior art devices result in suboptimum use of ultraviolet energy, are limited in flow rate, and result in wide variances in UVC dose amongst ingested target pathogens as a result of turbulence in the air flow.

### DISCUSSION OF THE PRIOR ART

Referring to U.S. Pat. No. 9,855,362 B2 to Rolf Englehard (hereinafter “the 362 patent”), the efficiency of the device is significantly lower than in the case of the present invention. Please refer to the applicable design principles enumerated in the Summary of Invention. FIG. 4b illustrates an embodiment that subjects the UVC radiation to numerous unnecessary reflections and attenuation. FIG. 8 illustrates an embodiment with unnecessary UVC losses in the upwind and downwind directions in conjunction with turbulent flow that results in unequal UVC exposure to any germs passing through. The chamber size is very small in relation to the UVC lamp capability. This results in an unnecessarily short UVC radiation path between reflections and thus limits the permissible air flow rate. This system as illustrated and described has a poor electrical efficiency and is only suitable for very small embodiments as are illustrated. The distinction between the “spiral flow” promoted in the 362 patent and the “free vortex flow” described in the present application could not be greater. The spiral flow described in 362 is intentionally turbulent and it is established in a non-isentropic manner, i.e., most of the kinetic energy imparted to the air flow is irrecoverable. This may be acceptable in a small device that does not rely on batteries for operation. It is very disadvantageous in a system designed for battery power as is the case with personal protective equipment (PPE). It is also disadvantageous for large scale heating, ventilation, and air conditioning (HVAC) air sterilizers where power requirements over the life of the equipment can be many multiples of the purchase price. Furthermore, inefficiencies, and the excess heat that they generate, may result in higher air condition loads and yet further power use and cost. In summary, the high intensity air purifier of the 362 patent has acceptable efficiency for small AC powered units, but its low UVC efficiency and low aerodynamic efficiency are a distinct disadvantages in the case of battery powered units or in the case of larger units passing hundreds or thousands of cubic feet per minute.

Referring to U.S. Pat. No. 10,039,852 B2 to Yi et, al (hereinafter “the 852 patent”), and referring again to the UVC air treatment chamber design principles enumerated below, the short distance across the UVC treatment chamber, relative to the cross sectional area of the UVC treatment chamber radiates any suspended pathogens over only a short distance. In order to achieve any given dose rate, measured for example in Joules/square centimeter, the air velocity and, therefore, the air flow rate must be limited. The illustrated design results in significant turbulence within the UVC treatment chamber. This results in some suspended pathogens receiving less than the average dose of UVC radiation, further limiting the acceptable air flow rate. The UVC treatment chamber configuration with the air flow impinging on a flat UVC emitter illustrated in the 852 patent results in unnecessary aerodynamic losses and requires a higher power blower than would be necessary in accordance with the present patent application. The high proportion of nonreflective area to reflective area in the UVC treatment chamber results in low UVC intensities in relation to the UVC power. The low UVC efficiency combined with the low aerodynamic efficiency combine to make the 852 patent less efficient than the UVC air sterilization system described in the present application.

### SUMMARY OF THE INVENTION

To better convey the advantages of the present invention, some basic UVC treatment chamber design principles should first be presented. This combination of principles appears to have been unknown or ignored in conjunction with much of the prior art. These principles are enumerated as follows:

1) UVC photons may be lost with every reflection. It is important to minimize the number of reflections. Sharp corners that would require extra reflections should be avoided.

2) The probability of a pathogen kill for any photon is proportional to the total travel distance of the photon within the UVC treatment chamber. A large chamber will maximize the probable travel distance by minimizing the required number of reflections.

3) High reflectivity is very important. If there were no photons lost to the air inlets and air outlets of the UVC treatment chamber, a UVC treatment chamber internal reflectivity of 98% would result in a UVC light intensity 5 times greater than in the case of a UVC treatment chamber internal reflectivity of 90%, using the same UVC source. The higher the UVC intensity within the UVC treatment chamber, the higher may be the air flow rate for a specified UVC dose (in Joules/square centimeter) to any pathogens.

4) UVC lost through the air inlet ports or air outlet ports of the UVC treatment chamber reduces the illumination intensity throughout the chamber. It is important that these losses be minimized. This can be done by minimizing inlet and outlet areas while avoiding excessive aerodynamic losses and noise.

5) Large scale turbulence within the UVC treatment chamber results in a high probability than some pathogens may be carried through without exposure to the specified UVC dose.

Suppression of large-scale turbulence within the UVC treatment chamber is critical to effective performance and to attaining a high probability of pathogen kill. Small scale turbulence over short distances associated with an array of entry nozzles has little effect on dosage probability distribution.

5) Aerodynamic losses may be minimized by incorporation of efficient nozzles to accelerate flow and the incorporation of efficient diffusers to decelerate flow.

6) Aerodynamic losses may be recovered by converting the kinetic energy at an exhaust port of limited size to useful kinetic energy in conjunction with the blower system. This may be accomplished with diffuser vanes or fan blades, for example. Fan blades may achieve their best efficiency point with the velocity vector of the air exiting the UVC treatment chamber, for example.

7) UVC photons that unavoidably escape from the UVC treatment chamber through an array of fine inlet nozzles, for example, may be put to good use in conjunction with a photocatalyst positioned upwind of the inlet nozzles. In this manner, these otherwise wasted photons are put to good use in powering photochemical decomposition of noxious substances in the air.

8) It is desirable in certain installations, such as in the case of ceiling fan replacement with a UVC air treatment system, to gather stratified heated and exhaled air from one plane (adjacent the ceiling in a restaurant, for example) and discharge it downward and out of the plane from whence it came. This results in more effective turnover and sterilization of all the air within a room. Returning stratified air to the same stratus from whence it came is much less effective in treating all the air within a room.

The present invention is a UVC air treatment system that is uniquely configured to suppress turbulence within the air treatment chamber and thereby provide all the processed air with precisely the same UVC dose. Providing the same dose to all the air is required in order to provide maximum germicidal effect for a given chamber volume, fan power level, UVC power level, acoustic noise level, system capital cost, and system operating cost. In addition to the benefits of suppressing turbulence, the unique configuration of the present invention maximizes the utilization of the lamp generated UVC energy by maximizing the mean free path between reflections off the chamber surfaces, while also minimizing the area of inlet and outlet ports where UVC radiation would otherwise be wasted. The inlet ports (nozzles) may be oriented at right angles to the UVC lamp and thus result in no loss of direct (not-yet-reflected) lamp energy. The result is that the total (direct plus reflected) UVC intensity is very high and is also nearly isotropic within the UVC treatment chamber without resorting to many high output UVC lamps with a high combined power consumption and maintenance cost. The unique configuration allows UVC photons to follow long pathogen-intercepting paths between efficient reflections, with minimal UVC loss at air inlets and air outlets. Reflection efficiency is maximized by incorporating highly reflective surfaces, such as expanded polytetrafluoroethylene (ePTFE) sintered polytetrafluoroethylene (PTFE), or polished aluminum, for example, throughout. The long photon paths between reflections are beneficial because attenuation (loss of photons) occurs upon each encounter with a reflective surface if those surfaces are highly reflective (98% efficient).

Turbulence suppression is preferably accomplished by first passing the air through filters which suppress any turbulence in the incoming air. From the filters the air is then accelerated in a uniform manner to one or more nozzles. The pressure gradient between the filters and the nozzles prevents flow separation and delivers air with laminar flow to each nozzle. In accordance with one embodiment of the invention, each nozzle delivers air to the treatment chamber in a precisely tangential manner without flow separation. The rotating air within the treatment chamber then behaves as free vortex flow as it migrates spirally inward within the treatment chamber. The radial pressure gradient within the treatment chamber prevents any flow separation. A light source consisting of one or more UVC lamps are situated to not interfere with the inherent spiral laminar flow pattern. A lamp or cluster of lamps may be coaxially situated in the center of the treatment chamber where they may be mounted to one face of the chamber, for example. Alternatively, tubular lamps may be inserted through the nozzles in an orientation parallel to the incoming air flow. In yet another embodiment, standard long tubular lamps may be installed behind flush quartz windows on either face of the generally cylindrical treatment chamber.

In accordance with another embodiment of the invention, large scale turbulence within the UVC treatment chamber may be suppressed by introducing the air through an array of perferations in a panel of UVC reflective material. The nozzles are preferably configured to have minimal entrance losses in order to maximize flow capacity, while introducing small scale turbulence in the plane normal to the flow direction such that the jets exiting the nozzles converge within a short distance to establish uniform velocity flow across the remainder of the UVC treatment chamber. The uniform velocity flow assures uniform UVC dosage for all air and pathogens traversing the UVC treatment chamber flow at a uniform velocity.

In accordance with a further aspect of the invention, a porous structure of photocatalytic material such as titanium dioxide may be positioned upwind of the nozzles at a distance of one nozzle spacing, for example, in order to illuminate the catalyst with UVC energy that would otherwise be lost through the nozzles. The provision of a stand-off distance between the nozzles and the catalyst structure allows for more uniform illumination of the catalyst than in the case of placement of the catalyst against the nozzle array. The stand off distance also allows air flowing uniformly through the photocatalytic structure to flow laterally between the photocatalytic structure and holes through the reflective inlet panel.

In accordance with a further aspect of the invention a flat or otherwise shaped panel of porous ePTFE may be used as a reflector and air inlet port of a UVC treatment chamber. In this manner air may be admitted with minimal loss of UVC energy.

In accordance with a further aspect of the invention, the air exiting the UVC treatment chamber may be directed through a streamlined narrow rectangular nozzle, circular nozzle or annular nozzle, for example, in order to minimize the loss of UVC energy through the air exit. The kinetic energy of the air passing through such a slot or nozzle may be largely recovered by means of a matching diffuser.

In accordance with a further aspect of the invention, one or more air filters may be used upwind of the nozzles, and upwind of the photochemical catalyst, if used. Such filters may include, but are not limited to, prefilters, HEPA filters, carbon filters electrostatic filters, and the like. One of the purposes of such filters is to remove any UVC opaque particles in which pathogens might be harbored, and to remove larger pathogens that may be relatively UVC resistant.

In accordance with a further aspect of the invention, prefilters, HEPA filters, carbon filters electrostatic filters, and the like may additionally, or alternatively, by used downwind of the UVC treatment chamber.

In accordance with a further aspect of the invention, at least one UVC treatment chamber inlet nozzle may be provided that is responsive to differential pressure across the nozzle. In this manner the nozzle opening may automatically adjust to preserve as much UVC light within the treatment chamber as possible for the instantaneously demanded flow rate. Flow rates may vary with time of day, building occupancy, nigh time blower noise constraints, personal oxygen demands, or, in the case of personal protective equipment, with breathing. In the case of a circular treatment chamber, the turbulence suppressing free vortex air flow pattern may be preserved for a period, even after air flow temporarily ceases to enter through the treatment chamber inlet nozzle(s).

In an analogous manner, a differential pressure responsive and UVC reflective exhaust valve may control air flow from the UVC treatment chamber. In this manner, UVC light loss through the exhaust port may be minimized.

In accordance with a further aspect of the invention, a UVC reflective propeller may be used to allow free flow of air through a port into or out of the UVC treatment chamber, while reflecting and containing most of the UVC that would have exited a simple open circular hole.

In accordance with a further aspect of the invention, a UVC reflective fan may be positioned within an entry or exit port of the UVC treatment chamber.

In accordance with a further aspect of the invention, the air outlet from the UVC treatment chamber may be through a porous UVC reflective material such as sintered PTFE. In this manner air may be discharged from the UVC treatment chamber with minimal loss of UVC energy from the UVC treatment chamber.

In accordance with a further aspect of this invention, other finely divided electrically insulating material that is resistant to UVC damage, such as sintered PTFE, bonded silica powder, non-woven silica fiber paper, or quartz fiber cloth, for example may be used as porous UVC reflectors for air flow into or out of the UVC treatment chamber.

In accordance with a further aspect of the invention, a porous UVC reflective material may be used as both a treatment chamber reflector and as a mechanical filter.

In accordance with a further aspect of this invention, the air outlet area may be configured as one or more nozzles to allow air to isentropically exit the UVC treatment chamber while minimizing the escape of UVC radiant energy. Such nozzles by their nature accelerate the air. The resulting kinetic energy in the air may be largely recovered by means of well-designed diffusers, an example being a cone of approximately 14 degrees total included angle. An annular nozzle may be provided in conjunction with an annular diffuser. Annular nozzles at the intersection of a cylindrical UVC treatment chamber and a generally planar end wall cause the required air exit area to be placed in a corner where some UVC energy would in any case have been attenuated by multiple reflections between cylindrical wall and end wall. This principal applies as well to the sides and end wall of a rectangular treatment chamber.

In accordance with a further aspect of the invention a UVC treatment chamber in accordance with the present invention may be used to sterilize the exhaust stream from a vacuum cleaner. For example, a spiral flow path treatment chamber may be fitted with an inlet connection that fits the outlet of a standard shop type vacuum cleaner and an outlet that fits the same vacuum cleaner hose. In this manner sterilized air may be conveniently directed.

In accordance with a further aspect of the invention, the UVC treatment chamber described herein may be incorporated into a vacuum cleaner. By this means, the exhaust air from the vacuum cleaner may be rendered less hazardous. Cyclone separators within a vacuum cleaner may also serve as UVC treatment chambers. Such an arrangement might use UVC transparent and wear resistant quartz cyclone housings in conjunction with ePTFE reflectors protected from dirt contamination and abrasion by the quartz housings. A periodic water spray could be used to remove dust from the interior of the cyclone housings. The required UVC lamps could conveniently be installed on the exterior of the quartz housing.

In accordance with a further aspect of this invention, the UVC air sterilizer described herein may be used to capture the air from a hand dryer in order to sterilize such air before reintroducing it to the room. Prior art hand dryers energetically disperse potentially infectious droplets and create a hazard for both the user and for bystanders.

In accordance with a further aspect of the invention, the UVC air sterilizer described herein may be used in conjunction with a toilet to prevent biohazardous mist from spreading throughout the room when the toilet is flushed. The spreading of this biohazardous mist is exacerbated by the convention of providing restroom ventilation fans near the ceiling despite toilet bowls being located nearer the floor. In many instances it may be more feasible to run electrical power to air sterilizers collocated with toilets than to run air ducts from each toilet to a central air sterilizer. Air sterilizers in accordance with the present invention may be located above a toilet tank, underneath a toilet tank, above or below a high capacity flush valve used in lieu of a tank, or behind a service wall, for example. Toilet seats with connection ports for forced ventilation are available and may be connected to the UVC air sterilizer of the present invention. Alternatively, in accordance with a further aspect of the invention, a toilet may be manufactured with an annular vent just beneath the rim of the toilet bowl in conjunction with a duct leading to a UVC treatment chamber and associated fan. The UVC treatment chamber could be integrated into the toilet.

In accordance with a further aspect of the invention, a propeller with little or no blade camber in conjunction with a shallow pitch angle may be made of or be coated with a UV reflective material. Such propellers may be mounted on bearings and used without a motor for the purpose of permitting air flow while simultaneously reflecting most of the UVC radiation that would otherwise escape out through the UVC treatment chamber air inlet or UVC treatment chamber air outlet. A simple polished aluminum propeller could be used for this purpose. Counter rotating reflective propellers could also be used in order to obstruct direct light paths between blades and reflect back at least a portion of the UVC radiation that would otherwise escape between blades parallel to the blade pitch angle.

In accordance with a further aspect of this invention, the UVC air sterilizer described herein may be used to sterilize air used for an air curtain in a doorway, for example. In this manner, the sheet of moving air not only serves to keep pathogens from crossing the air curtain, the sterile blowing air also serves to displace any pathogen contaminated air in the vicinity. The resulting sterilized curtains of air may be used to create virtual walls between dinner tables at a restaurant. between aircraft passengers, between aircraft passengers and crew members, between medical staff and patients, between a checkout clerk and customers or between the passengers and the driver of a taxi, Uber or Lyft automobile, for example. Numerous other applications are made possible by the unique high volume air sterilization capacity of the UVC air sterilizers in accordance with the present invention.

### BRIEF DESCRIPTION OF THE DRAWINGS

FIG. 1a is an isometric rendering of the treatment chamber of the present invention showing inlet nozzle streamlines 29 and free vortex streamlines 30, shown in FIG. 1b, within UVC treatment chamber 1, shown in FIG. 2a, calculated by computational fluid dynamics (CFD) software.

FIG. 1b is a cutaway view of the rendering of FIG. 1a.

FIG. 2a is a cutaway rendering of one embodiment of the present invention.

FIG. 2b is an exemplar velocity diagram illustrating the fan inlet and outlet velocity triangles associated with the embodiment of FIG. 2a. It should be noted that this energy recovering design principle applies to radial flow, mixed flow, and axial flow fans. In each of these cases the blade inlet and outlet angles can be made to match. From hub to shroud along the entire length of the leading edge of the blades, the free vortex flow exiting the UVC treatment chamber and in each case the blades may be shaped to result in very low exit velocity tangential components. Good design practice will result in uniform axial velocities as well. When these fan exit conditions are combined with an efficient conical diffuser the overall aerodynamic efficiency of the system will be far better than that of the prior art.

FIG. 3 is a cutaway view of an embodiment of the present invention incorporating a fan that uses the UVC chamber air inlet nozzles as upwind guide vanes and incorporates a tubular stand for air conveyance. The tubular stand allows warm exhaled, potentially germ laden air to be gathered near the ceiling and delivered near the floor without blowing it directly onto people. The use of a relatively narrow stand permits installation of the associated UVC air sterilizer where floor space might otherwise be insufficient.

FIG. 4 is a cutaway view of an embodiment similar to FIG. 3 except with a fan assembly that includes upwind guide vanes.

FIGS. 5a, 5b, 5c, and 5d illustrate the function of various numbers of inlet nozzles in conjunction with one embodiment of the present invention.

FIGS. 6a and 6b illustrate an embodiment of the present invention in conjunction with a single elongated inlet nozzle, an axial flow fan without upwind guide vanes, and a flexible exhaust duct.

FIG. 7 illustrates an embodiment of the present invention incorporating two elongated inlet nozzles shaped to prevent the escape of UVC radiation.

FIGS. 8a, 8b, 8c, and 8d illustrate an embodiment of the present invention that incorporates a pressure responsive inlet nozzle 3a-d with variable opening 79 and a pressure responsive exhaust port valve 26 for the purpose of minimizing UVC radiant energy leakage at reduced air flow rates. Free vortex flow 46 is illustrated in UVC treatment chamber 1. Filter 5 minimizes contamination of treatment chamber 1 and suppresses turbulence in the incoming air.

FIGS. 9a, 9b, 9c, and 9d illustrate a free vortex UVC treatment chamber scaled down in size for use as a respirator cartridge.

FIG. 10 illustrates a UVC air sterilizer configured to replace a standard light bulb.

FIGS. 11a, 11b, 11c, 11d, 11e, 11f, and 11g illustrate an ultraviolet air sterilizer configured to replace a ceiling fan and associated lights.

FIG. 12 illustrates an example configuration of a reflective air inlet plate in conjunction with the present invention.

FIG. 13 illustrates the shape of a single jet from a single smooth and non-serrated nozzle in a reflective air inlet panel.

FIG. 14 illustrates one embodiment of a UVC air sterilizer in accordance with the present invention.

FIGS. 15a, 15b, and 15c illustrate, respectively, a sectional elevation view, a perspective view, and a detailed view of the associated reflective air inlet panel of a UVC air sterilizer in accordance with the present invention.

FIGS. 16a, 16b, and 16c illustrate a sectional elevation, a cutaway perspective view, and a detail section of one embodiment of a UVC air sterilizer in accordance with the present invention.

FIGS. 17a and 17b illustrate the incorporation of a UVC air sterilizer in accordance with one embodiment of the present invention into an electric hand dryer.

FIGS. 18a, 18b, and 18c illustrate a plan view, side view, and a perspective view of a UVC air sterilizer in accordance with the present invention that is configured for installation in a suspended ceiling.

FIG. 19 shows an embodiment of the present invention configured to sterilize pathogens otherwise dispersed during the flushing of a toilet.

FIG. 20 shows a UVC air sterilizer in accordance with one embodiment of the present invention used to capture any pathogens exhaled by persons seated at a table for example.

FIG. 21 shows a UVC air sterilizer in accordance with the present invention installed in conjunction with an air curtain.

FIG. 22 shows a UVC air sterilizer in accordance with the present invention incorporated into a vacuum cleaner.

### DETAILED DESCRIPTION OF THE PREFERRED EMBODIMENTS

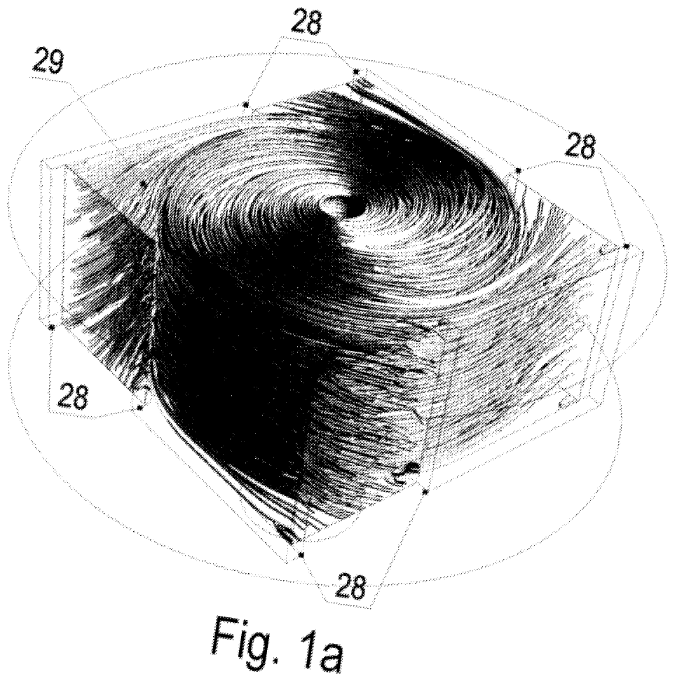

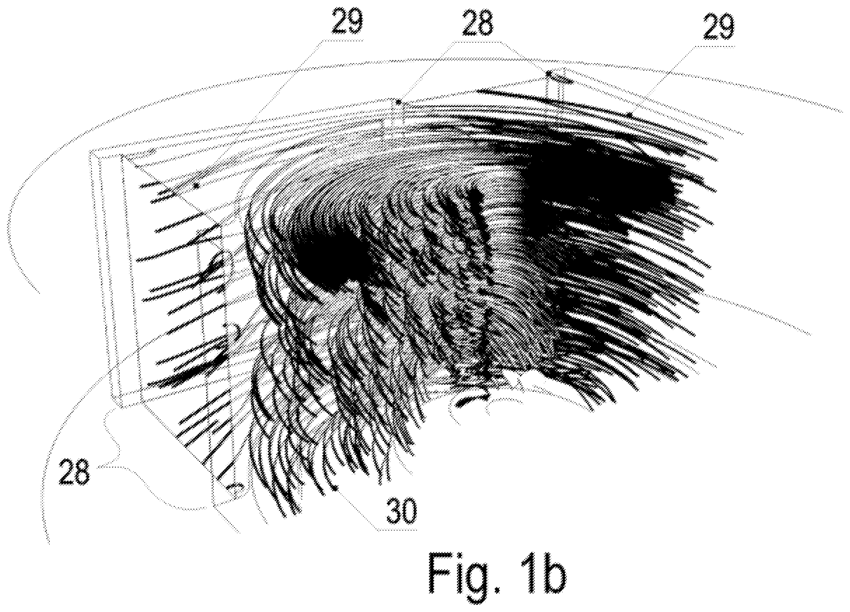

Referring to FIGS. 1a, 1b, and 2a, the turbulence suppressing free vortex flow unique to this invention is illustrated. Note that the purpose of these figures is to illustrate the air flow streamlines. Air enters the device at air inlets 28. Some of the detail of the device has been omitted from these views in order to more clearly reveal the converging inlet nozzle streamlines 29 in conjunction with the free vortex streamlines 30 in the UVC treatment chamber 1. UVC treatment chamber 1 is labelled in FIG. 2a.

Referring to FIG. 2a, air enters the UVC treatment chamber 1 through filters 5a, 5b and 5c then nozzles 3a, 3b, and 3c The air particles in UVC treatment chamber 1 are bombarded by UVC rays 38 that are being emitted by the UVC lamp 9. After transiting the chamber in a free vortex, the air exits through diffuser 11 Fan 12 is preferably optimized to efficiently engage the swirling air exiting UVC treatment chamber 1 and discharge it axially down diffuser 11 in accordance with the velocity triangles illustrated in FIG. 2b.

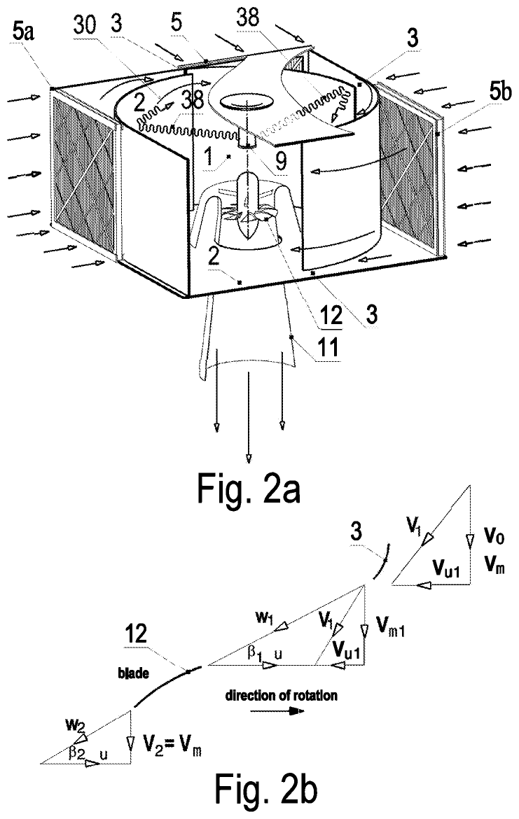

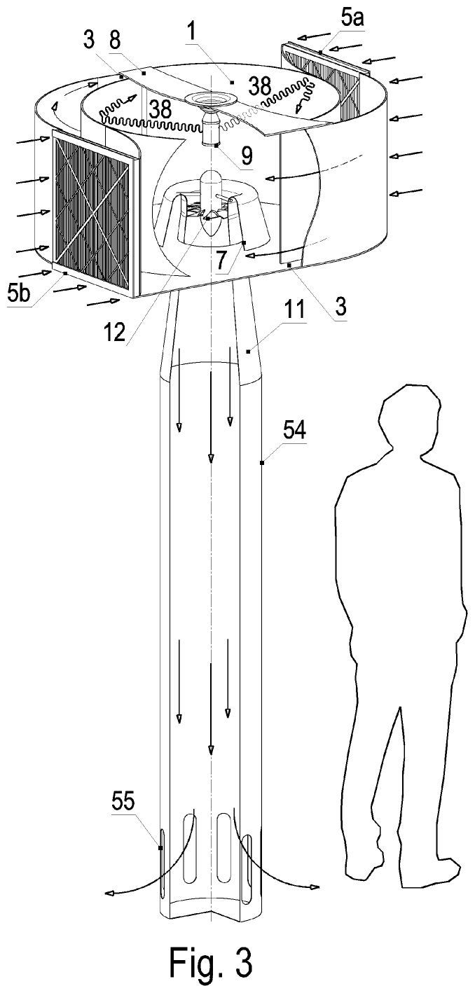

Referring to FIGS. 3 and 4, an embodiment of the present invention is illustrated that incorporates two inlet filters, 5a and 5b From the inlet filters 5a and 5b, the air accelerates isentropically through nozzles 3a and 3b into generally cylindrical UVC treatment chamber (1 where it follows an organized turbulence free accelerating flow path to fan 12 at the exit. Fan 12 may be a commercial off-the-shelf fan, or it may be a fan designed to recover the tangential velocity in the exiting free vortex flow in accordance with the example velocity triangles of FIG. 2b. The exiting flow encounters the leading edges of the fan blades already having tangential velocity Vul. The fan may be designed in accordance with conventional design practice and confirmed using computational fluid dynamics(CFD) software such that the outlet flow of the blade trailing edges is substantially axial. In this manner the kinetic energy that was provided to the air at the nozzles 3a and 3b is partially recovered. The balance of the kinetic energy imparted at nozzles 3a and 3b may be largely recovered in a diffuser 11. Guide vanes 15 may be used to improve flow through the fan 12. The air exits the diffuser 11 and flows down the vertical duct 54 to exit through the floor level exhaust ports 55.

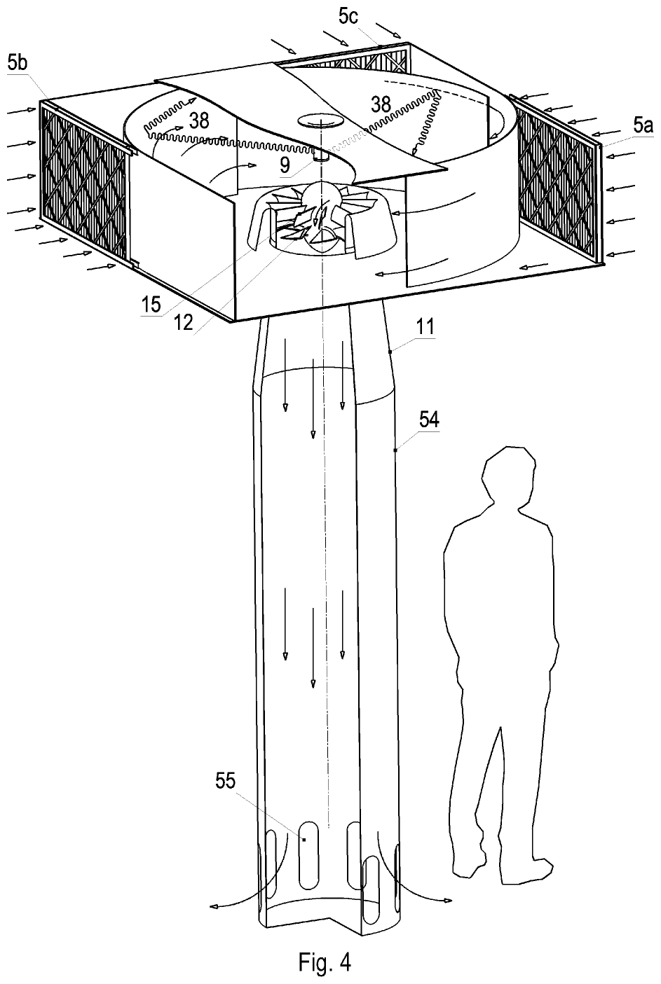

Referring to FIGS. 2a, 3, 4, 5a, 5b, 5c, 5d, UVC air sterilizers in accordance with several embodiments of the present invention are illustrated. As can be seen, various numbers of filters and nozzles may be used in accordance with power budget, available space, and desired air flow rate. Air is fed into the generally circular UVC treatment chamber 1 through nozzles 3a, 3b, 3c, and 3d. Air is supplied to each of these nozzles through air inlet filters 5. The air inlet filters 5 help dampen any turbulence in the air entering nozzles 3a, 3b, 3c, 3d and occlude contaminants from UVC treatment chamber 1. The filters 5a, 5b, 5c also improve germicidal effectiveness of the system by trapping particles and droplets large enough to shade germicidal particles from the UVC. The germicidal effectiveness of the UVC air treatment system on air transiting the UVC treatment chamber 1 is proportional to the UVC intensity within the UVC treatment chamber 1 multiplied by the transit time through the UVC treatment chamber 1. The effectiveness also depends on the uniformity of UVC intensity and on the transit time. The UVC intensity is a function of the intensity of the UVC light source which consists of at least one UVC lamp 9, the reflectivity of the interior of the UVC treatment chamber 1, the uninterrupted photon path length between reflections, and the rate at which UVC radiation is lost from the UVC treatment chamber 1 to the inlet nozzles 3, and the outlet port 46. FIGS. 5a, 5b, 5c, and 5d illustrate the use of various quantities of filters and inlet nozzles. The number of nozzles may be easily adjusted to control the exterior shape and dimensions of the UVC air sterilizer, as well as to accommodate the direction from which air is drawn into filters 5.

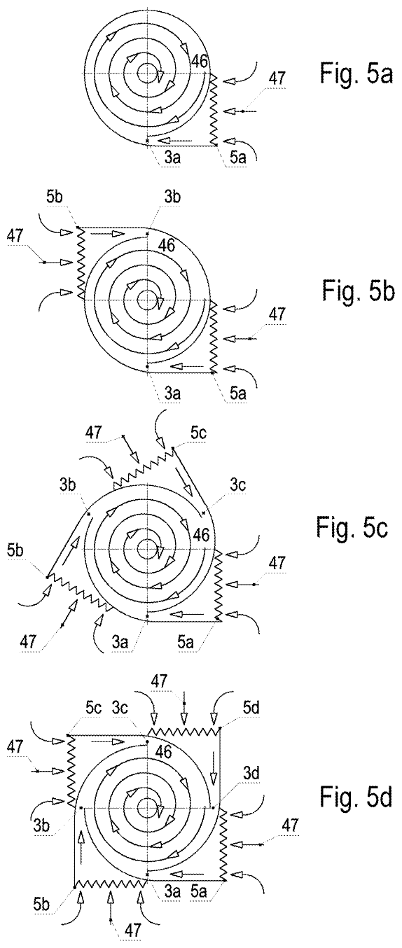

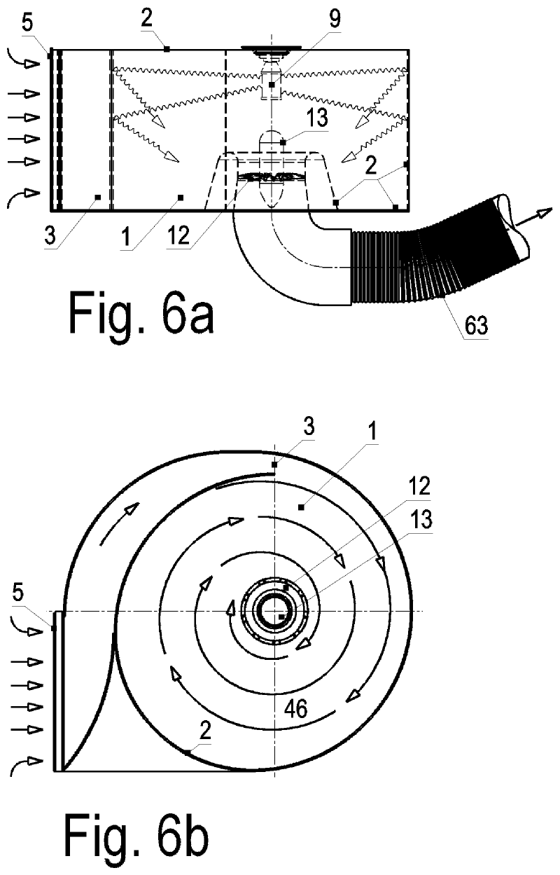

Referring to FIGS. 6a, 6b, and 7, embodiments of the present invention are illustrated wherein the flow path between filter 5a-5c and nozzle 3a-3d (is configured to allow the smooth laminar flow of air from filters 5a-c to nozzle 3a-d, while obstructing the path for otherwise escaping UVC radiation. UVC radiation bounces of reflective points 47, 48, 49 and 50. UVC radiation reflecting off of point 50 has no direct path to the filter 5 (5a, 5b, 5c, etc). UVC radiation escaping from UVC treatment chamber 1 is absorbed by the non-reflective surfaces at points 43, 44, and 45 before it can reach filters 5a-5d. Referring to FIGS. 6a and 6b, UV treatment chamber 1 is surrounded by UVC reflective surfaces 2. Motor 13 drives fan 12. Air enters UVC treatment chamber 1 through nozzles 3a-3d from which it behaves as free vortex flow as it approaches the fan 12. After the fan, the air exits through duct assembly 63.

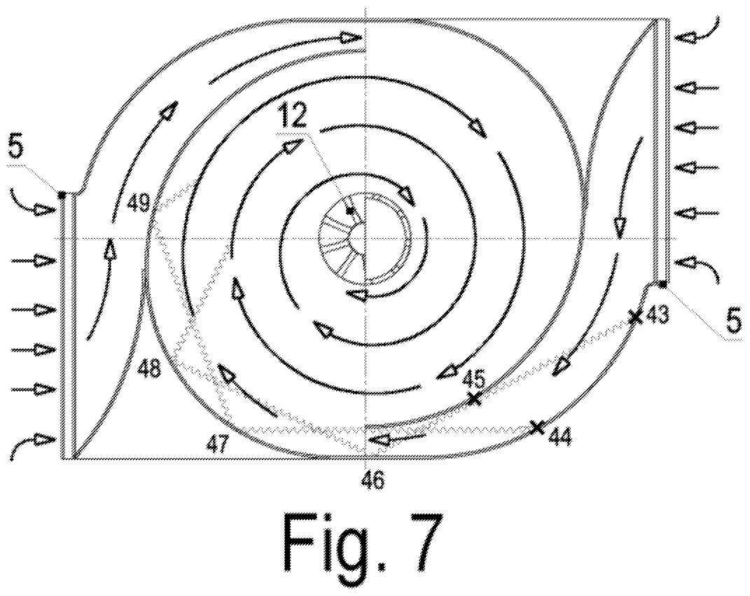

Referring to FIGS. 8a, 8b, 8c, and 8d, a UVC air sterilizer is illustrated that allows the escape of UVC radiation at the inlet and nozzle 3a and the exhaust port 26 to be automatically adjusted to accommodate the instantaneous air flow rate. In the case of a personal respirator, air flow due to inhalation and exhalation occurs cyclically. This is also the case with pulmonary ventilators in conjunction with which there may be a need to disinfect exhaust air. The embodiment illustrated in FIGS. 8a, 8b, 8c, and 8d maximizes UVC intensity for any given flow rate and also continues to sterilize the air within the UVC treatment chamber 1, even when the valves are shut between breaths. The UVC lamp 9 may be switch off whenever the ports are closed in order to conserve battery power, for example. This would preferably be done with an LED UVC source rather than with a low pressure mercury lamp requiring startup time.

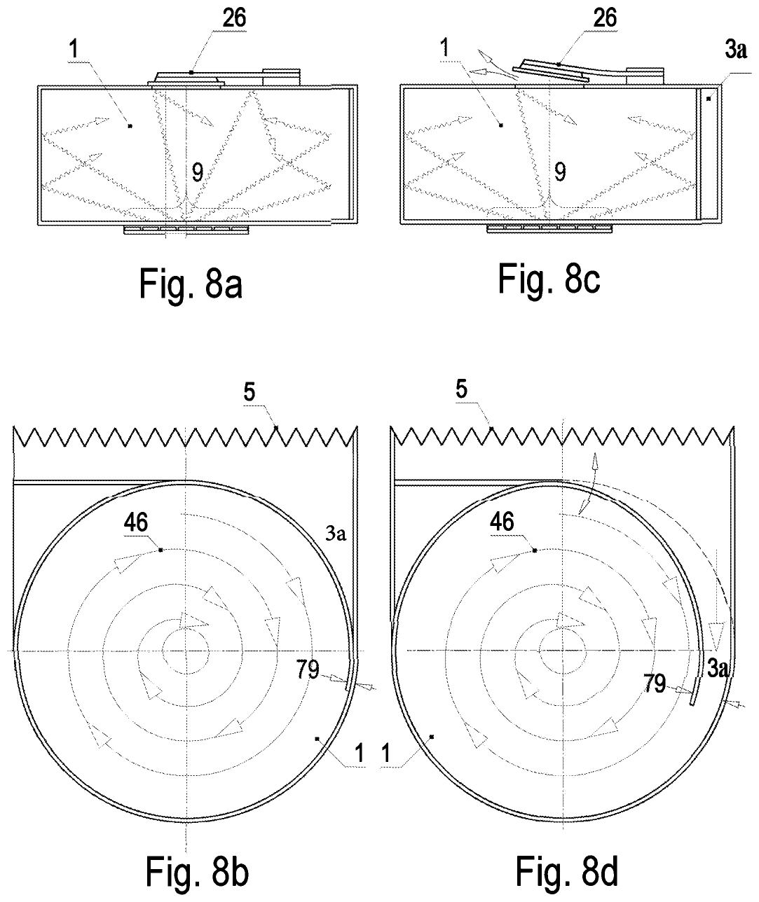

Referring now to FIGS. 9a, 9b, 9c, and 9d, a UVC treatment chamber 1 is shown configured and sized to fit a cartridge style respirator as personal protective equipment (PPE). Circumferential filter 6 cleans air before it enters curved baffles 7 that guide the air into UVC treatment chamber 1. Solid state UVC lamp 9 illuminates the interior of UVC treatment chamber 1. Connector 80 fastens the UVC treatment chamber 1 to a respirator mask or the like.

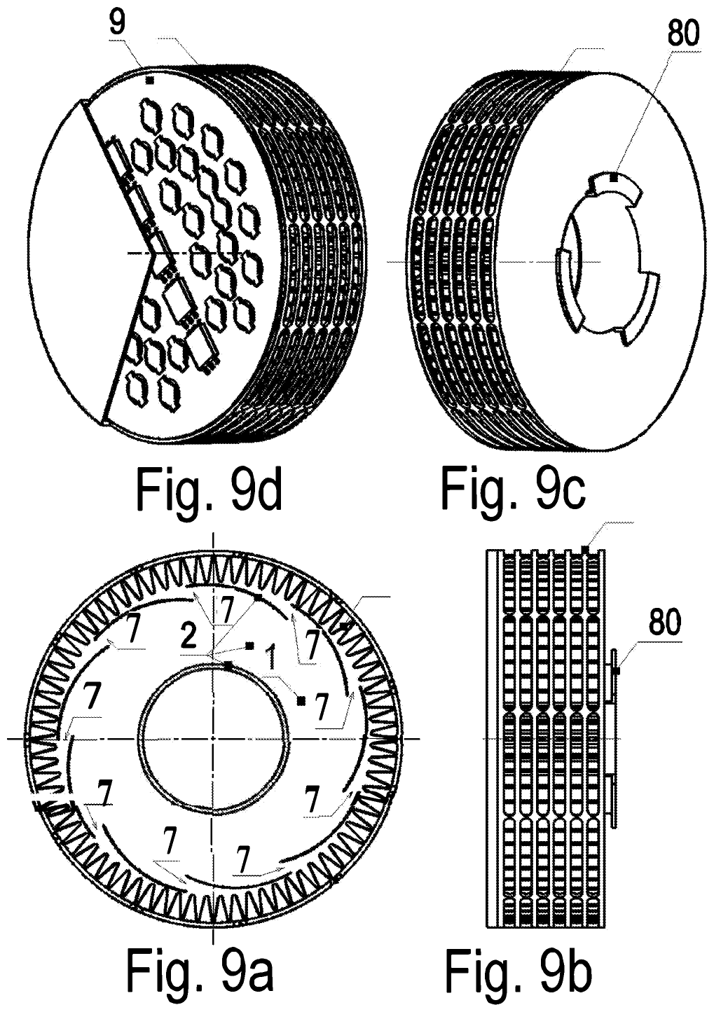

Referring now to FIG. 10, another embodiment of a UVC air purifier is shown. This configuration is designed to be conveniently installed as a replacement for an ordinary light bulb in an overhead socket 37. Such sockets are generally designed and wired in accordance with building codes that were written before the development of LED lights which draw much less power. Consequently, extra power is available at such sockets if LED lamps are used instead of the traditional tungsten filament incandescent light bulbs. The illustrated configuration screws directly into a standard existing light bulb socket 37 that is mounted to the room ceiling 36 with a terminal box 24. No tools are required. The UVC sterilizer housing is made up of a top panel 7, a bottom panel 8 and side panels 42. A threaded male electrical receptor is mechanically fastened to the UVC air sterilizer housing and is wired to a lamp socket below, to an infrared receiver 58 and control relay, and in turn to a replacement visible light LED lamp 22 below. An infrared remote control 57 may be used to turn on and off the LED lamp 22, to turn on and of the UVC air sterilizer, and to control fan speeds on the UVC air sterilizer. Inlet filter 5a-5c feeds air to a nozzle 3a-3d from which it discharges air into UVC treatment chamber 1. The air in the UVC treatment chamber 1 flows a free vortex flow path 46 until it enters fan 12. Fan 12 is preferably designed such that the swirling air exiting the UVC treatment chamber 1 impinges the leading edge of the fan blades without flow separation and leaves the trailing edge of the fan blades with little or no angular momentum. The upper surface of the fan blades may incorporate a UVC reflective material on the upper surface. Baffles 16 prevent escape of UVC radiation from the device and UVC energy irradiation of persons below.

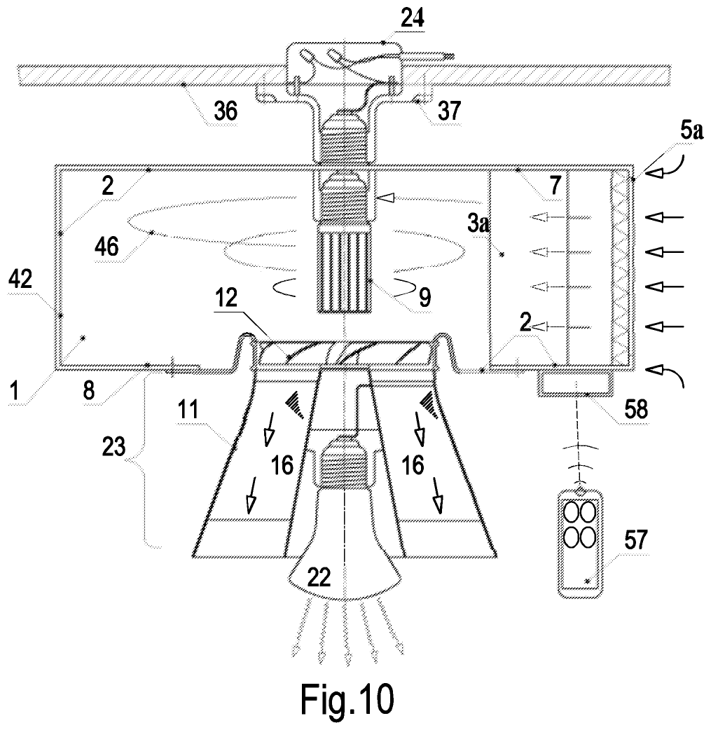

Referring now to FIGS. 11a, 11b, 11c, 11d, 11e, 11f, and 11g, another embodiment of the present invention is shown. This is a UVC air sterilizer configured to replace a ceiling fan 21. Ceiling fans are common in restaurants where they may exacerbate the spread of disease such as COVID-19. Replacement of existing ceiling fans with this embodiment of the present invention maintains air circulation, with sterilized air, and provides replacement lighting for any lighting lost with removal of ceiling fans. FIG. 11a illustrates a typical ceiling fan installation. The electrical terminal box 24 for a ceiling fan 21 is typically reinforced by a steel bracket to take the weight of the fan. Such electrical terminal boxes 24 are well suited to support this embodiment of the UVC air sterilizer in accordance with the present invention. FIG. 11b shows the exposed electrical box after ceiling fan 21 has been removed to allow installation of the UVC air sterilizer in accordance with this embodiment of the present invention. FIG. 11c shows the housing 41, including filter slots 5 and UVC treatment chamber 1 fastened to and wired to the pre-existing electrical terminal box 24. This fastening may be accomplished with simple hand tools. Referring now to FIG. 11d, once the housing is secured to and wired into the ceiling fan electrical box 24, the fan and lamp assembly 23 may be inserted from below and secured to the housing 41 with fasteners. Referring now to FIG. 11e, lamps 22 replace any lamps removed the ceiling fan 21 or obscured with installation of housing 41. Air enters through filters 5 from which it flows through nozzles 3a, 3b, 3c, and 3d. The air exits the UVC treatment chamber 1 through fan 12. UVC radiation is provided by UVC lamp 9. Louvre 20 may be adjustable in both azimuth and elevation for maximum comfort of persons seated or standing below. Control may be via hardwiring originally installed for the original ceiling fan or by means of infrared remote control, for example.

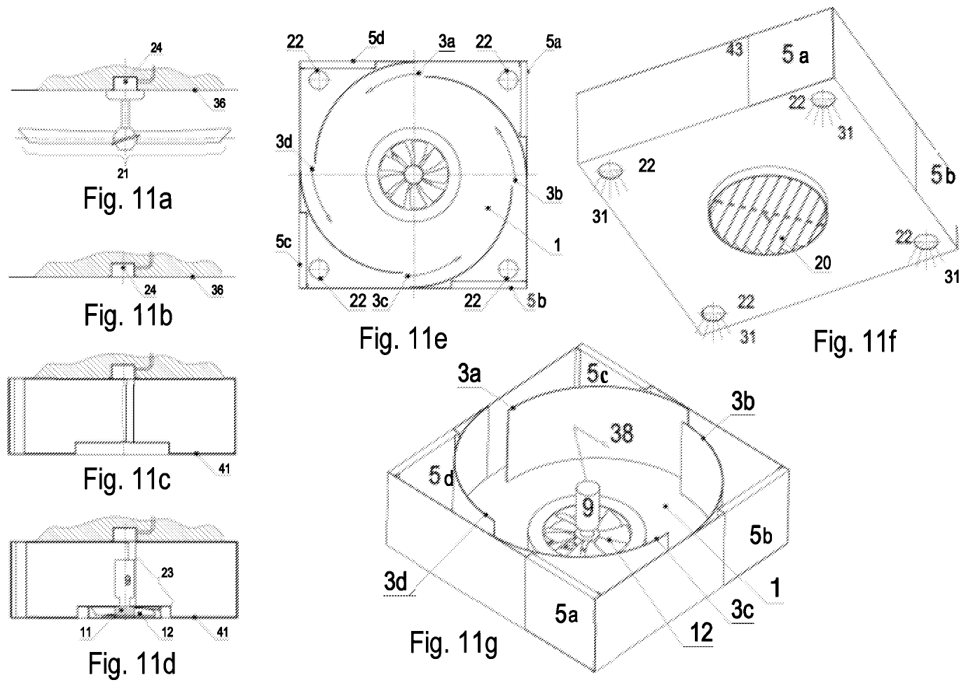

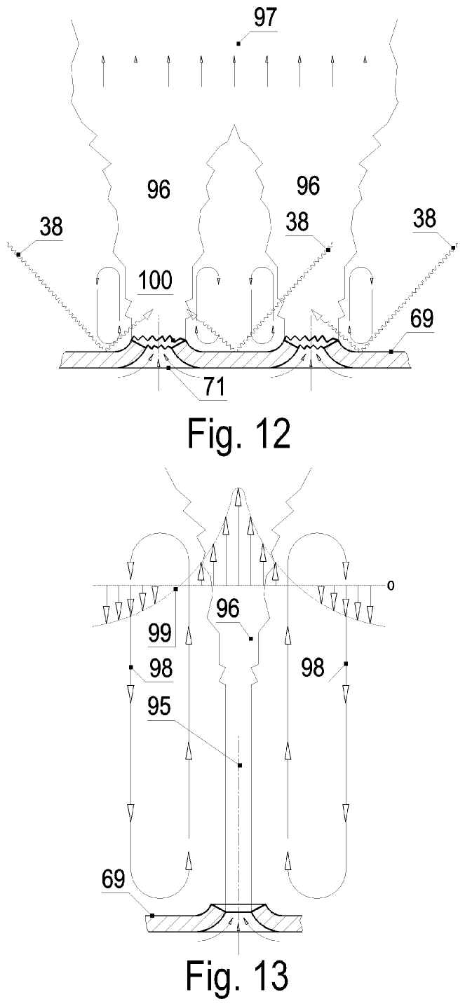

Referring now to FIGS. 12, 13, 14, 15a, 15b, 15c, 16a, 16b, and 16c, a different group of embodiments of UVC air sterilizer is shown. These are similar to the aforementioned embodiments that use a free vortex chamber to suppress turbulence insofar as they are also carefully configured to suppress turbulence and to minimize the escape of UVC radiant energy at the UVC treatment chamber inlets and outlets. Air is admitted to the UVC treatment chamber 1 through a porous or perforated panel that allows sufficient air to be admitted while simultaneously blocking the excessive escape of ultraviolet radiation. In the case of a perforated panel, the holes are preferably chamfered or bell mouthed on the upwind side in order to minimize pressure drop across the panel. The holes may also be serrated or roughened in order to induce a controlled degree of turbulence in the jets emerging from the downwind surface of the panel. By this means the jets merge within a short distance of the perforated panel and coalesce to form a flow of uniform velocity. This uniform velocity results in uniform residence time in the UVC treatment chamber 1. As in the case of the free vortex UVC treatment chamber 1, the UVC intensity in each direction along all three axes is very uniform on account of low losses at the UVC treatment chamber inlets and outlets. The combination of isotropic and uniform UVC illumination throughout the UVC treatment chamber 1, in combination with the uniform residence time in the UVC treatment chamber 1 provide a more uniform UVC dosage for any entrained or suspended pathogens than is provided in accordance with any of the prior art.

The best embodiment of the present invention for any given application depends on available space, required orientation of inlets and outlets, and required electrical efficiency. The free vortex UVC treatment chamber 1 provides the best electrical efficiency due largely to the nearly isentropic air flow. It may be the best choice for battery powered applications such as vacuum cleaners or battery powered personal protective equipment (PPE). It is also likely the best choice if air is to be drawn through or exhaled through the UVC chambers by the user and without electrical power. The free vortex UVC treatment chamber 1 is also a likely first choice for very high flow rate HVAC applications where space is not an issue. This is due again to low pressure losses across the system and lower blower power requirements.

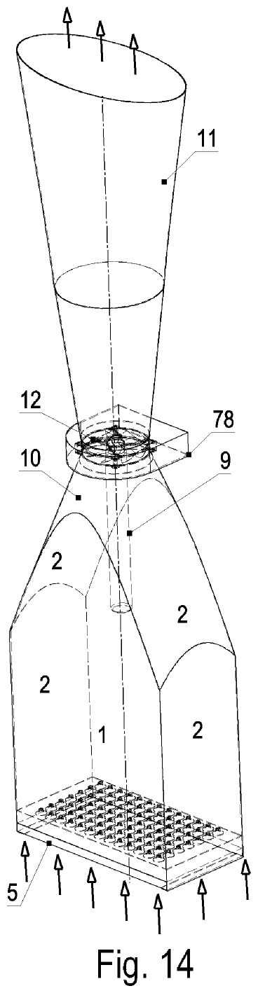

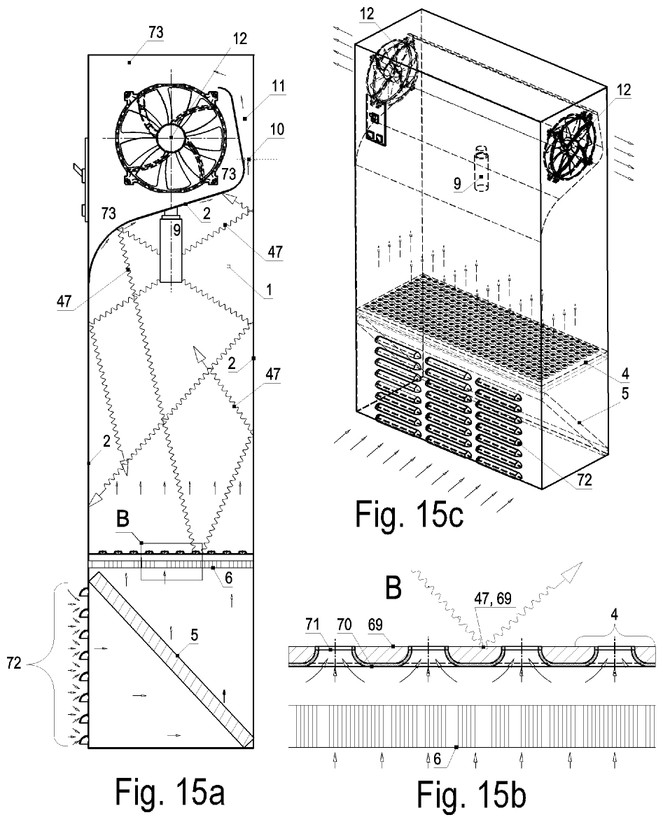

For applications where straight through air flow is preferred or a compact form factor is better suited, a UVC treatment chamber with a UVC reflective porous air inlet panel may be better. This embodiment may take the form of a tube, in which case it may be conveniently fitted with commercially available round filters and round duct blowers. The resulting UVC treatment chamber shape is very similar to that of the free vortex UVC treatment chamber. The UVC treatment system may vary in shape according to the application and may be for example a venturi shape as in FIG. 14, or it may be rectangular in shape as illustrated in FIGS. 15a, 15b, and 15c.

Referring to FIGS. 12, 13, 14, 15a, 15b, 15c, 16a, 16b, and 16c, an alternative method of suppressing turbulence within the UVC treatment chamber 1 which is illustrated wherein a “porous reflective panel” is provided through which air flows into the chamber. This panel serves the purpose of reflecting as much UVC as possible back into the UVC treatment chamber 1 while generating as little pressure drop as possible. The porosity may be over a range of scales. For example, it may take the form of a large number of closely spaced (6 mm for example) fine (2 mm diameter for example) perforations into which the air may enter isentropically. It should be noted that the exit losses from the perforations are largely unavoidable, but may be traded in the design against UVC radiant energy losses to achieve an acceptable compromise that is nonetheless superior to the prior art. The perforations may be nearly isentropic but should impart just enough fine scale turbulence to cause the jets to quickly merge together downwind of the nozzles perforations to form a single air mass 97 of uniform velocity. This is illustrated in FIG. 12 wherein air flows through perforations 71 and is disturbed by serrations 100.

Referring to FIG. 15b, the reflective surface of the perforated panel 4 may be ePTFE or sintered PTFE, for example. Given that these materials are relatively soft, a support structure 70 of aluminum, for example, may be provided. Alternatively, the reflective and turbulence suppressing panel may be comprised of highly pervious sintered PTFE or of bonded or sintered PTFE filaments, for example. Suppressing the turbulence and equalizing the UVC chamber inlet velocity provides each air parcel and each entrained pathogen the same chamber transit time and the same UVC dose. In this instance the panel provides approximately 89% reflective area despite the holes. Using a reflective material such as expanded or sintered PTFE with a UVC reflectance of 98% results in a net panel reflectance of approximately 87%. The porosity required for air entrance may alternatively be provided by the use of sintered beads or bonded filaments of PTFE, for example. In this case both the air pressure drop (aerodynamic energy loss) and the reflectivity (UVC energy not lost) would be expect to be somewhat higher.

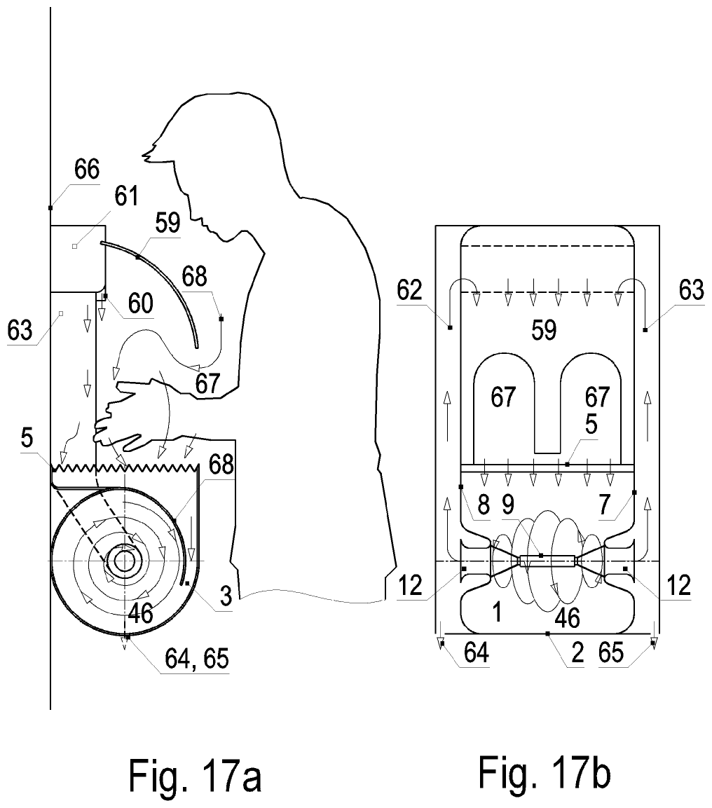

Referring to FIGS. 17a and 17b, an embodiment of the UVC air sterilizer in accordance with the present invention is illustrated that sterilizes the air that has been used to dry the users hands, while also providing a jet of sterile air from 60 (this needs to be called something other than nozzle as we have other nozzles 3 and we can only have one) for hand drying. The arrangement provides for venting some of the sterilized air at vents 64 and 65 in order to maintain an air deficit and a slight negative pressure within the shielded hand drying compartment under shield 59. Prior art hand dryers tend to blow potentially pathogen contaminated droplets all over the room where they may be inhaled by or contaminate the eyes of either the used or other persons nearby. The hand dryer configuration in accordance with the present invention provides a safe alternative to prior art hand dryers without resorting to paper towels that have their own pathogen spreading potential.

The lamp intensity can be selected within a wide range. The costs of higher UVC lamp power include original cost, replacement cost, electricity use, and any power needed to air condition the space to which heat is added by the UVC source. In general, it is desirable to obtain the greatest germicidal effect using the least amount of electrical power. In fact it is the essence of the present invention to accomplish that goal more perfectly than does the prior art. Germicidal effect might be measured in terms of flow rate (kg of air per second) times dose rate (Joules per square meter) divided by the combined power consumption of the fan(s) plus the UVC lamps. The dose rate should be based on the dose rate for the least treated parcels of air that make it through the UVC treatment chamber. In the case of prior art UVC treatment chambers, air turbulence within the treatment chamber results in great disparities in residence time in the chamber for various parcels of air. In the case of prior art, the least treated air typically has a substantially lower dose rate than the median treated air because of differences in velocity transiting the treatment chamber and differences in path length transiting the treatment chamber, as well as differences in UVC intensity throughout the UVC treatment chamber. Each of the illustrated and disclosed embodiments of the present invention incorporate countermeasures to large scale turbulence.

One of these countermeasures is the use of an accelerating free vortex flow path. A principal advantage of this configuration is that it is nearly isentropic because pressure energy of the incoming air is converted to kinetic energy in a nearly lossless manner through one or more efficient nozzles.

Referring to FIG. 14, air enters through a filter and air inlet panel secured in slots 38. UVC treatment chamber 1 features internal UVC reflective surfaces 2 illuminated with UVC lamp 9. Air exit converging passage 10 efficiently accelerates the air into fan 12. The air exits fan 12 into axial diffuser 11. Bracket 78 may be used to support the entire structure near its heaviest point where the fan and lamp are supported. This embodiment is more efficient than the prior art designs but somewhat less efficient than the embodiment of this invention incorporating the free vortex UVC air treatment chamber. It is potentially too long to use in some applications, however.

A functionally similar but shorter embodiment is illustrated in FIGS. 15a, 15b, and 15c. Air enters through louvre 72, passes through filter 5, then passes through TiO2 (Titanium Dioxide) honeycomb photocatalyst 6. It then flows a short distance to reflective inlet panel 4. The non-zero distance between the honeycomb photocatalyst panel 6 and the inlet panel 4 is to allow UVC radiation that leaks through the holes in the air inlet panel to defocus or spread out to more uniformly illuminate the honeycomb photocatalyst panel 6. It this way the “leaked” UVC radiation is not wasted. The UVC treatment chamber is illuminated by UVC lamp 9. UVC radiation escape out of the air exit is limited by linear nozzle 10. The kinetic energy imparted to the air at converging passage 10 is largely recovered by diffuser 11. Diffuser 11 and plenum 73 are preferably coated with a UVC absorbing material in order to prevent UVC exposure of bystanders. Fans 12 may be provided at each end of plenum 73, for example. The fans may be of standard design or they may be optimized to utilize the residual angular momentum with which the air enters plenum 73. In this case the fans would optimally rotate in opposite directions relative to their own inlets and outlets, but rotate in the same absolute direction as each other, in this case clockwise in view 15a.

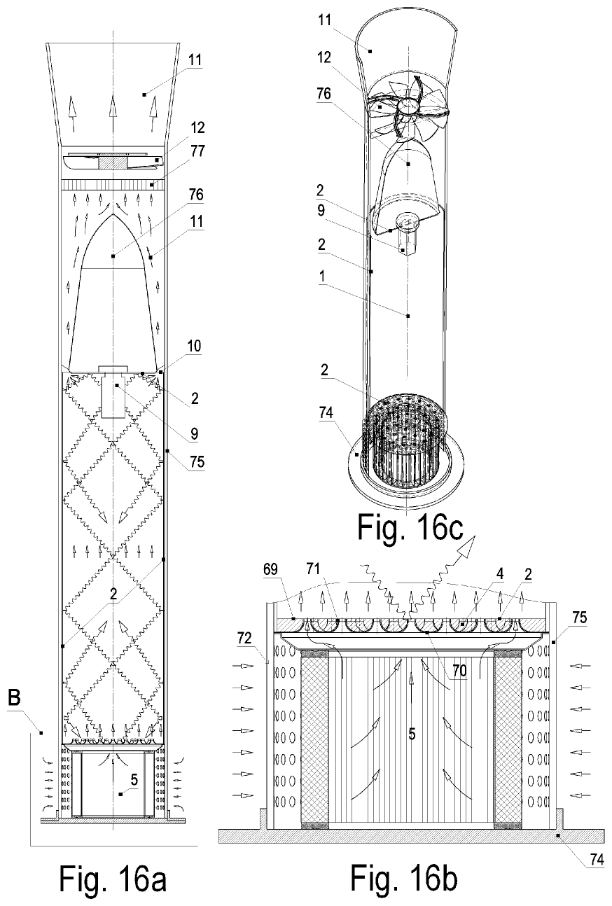

Referring now to FIG. 16 (a, b, or c), base 74 has at least one opening and supports pipe housing 75, which is perforated at its lower end to feed air to a standard HEPA shop vac filter 5. After leaving filter 5 the air passes through reflective air inlet panel 4 with reflective surface 2 and supporting structure 70. Pipe housing 75 may be made of PVC, aluminum, or galvanized sheet steel, for example. The pipe is preferably lined with a high UVC reflectivity liner such as expand PTFE (ePTFE) or sintered PTFE, for example. It should be noted that this design may accommodate more than one filter stacked end on end in order to accommodate higher air flow rates. The air transits the UVC air treatment chamber and then enters converging passage 10. Converging passage 10 is sized to allow reasonably low pressure drops with minimal UVC loss. Air exit fairing 76 acts in conjunction with the pipe housing 75 to create annular diffuser 11. The air then passes through photocatalyst panel 77 that is illuminated by UVC radiation that leaks through the nozzle 10. Photocatalyst 77 panel may incorporate UVC light baffles on its downwind surface to prevent escape of UVC radiation. Alternatively, air pervious UVC radiation baffles may be separately provided between photocatalyst panel 77 and fan 12. Fan 12 propels the air. Diffuser 11 reduces pressure drop through the system. An axial fan is shown. The fan may be located external to the system in either the upwind or downwind direction.

Other types of fans may be used, such as mixed flow or radial fans. In the case of installation within a motor vehicle or aircraft, for example, the air source may be taken from the impinging flow of air on the exterior of the vehicle or aircraft.

Referring now to FIGS. 17a and 17b, an electric hand drier mounted to the wall 66 in accordance with one embodiment of the present invention is illustrated. Blowers 12 circulate air through UVC treatment chamber 1 and discharge it into air channels 62 and 63. Most of the air flows upward into volume 61 from which it exits through nozzles 60 to dry the users hands. in order to maintain a slight negative pressure behind transparent hood 59. This allows makeup air 68 to enter which minimizes the escape of poetically contaminated droplets, keeping in mind that the advantage of this hand dryer system over the prior art is its UVC sterilization of the water shed off of the users hands.

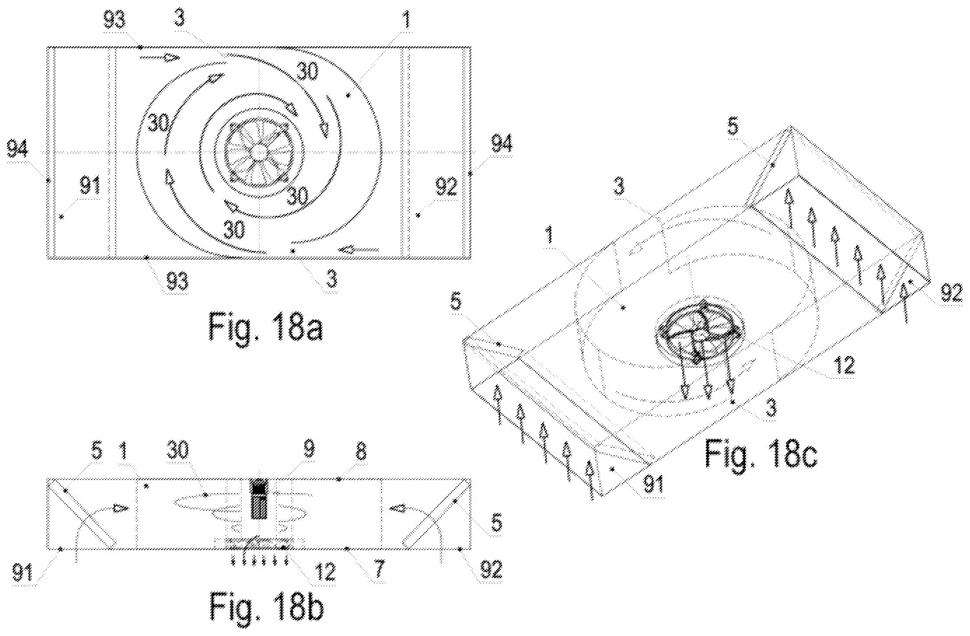

Referring to FIGS. 18a, 18b, and 18c, a UVC air sterilizer is shown that is configured for convenient installation in a suspended ceiling. Air intakes 91 and 92 feed air to inclined filters 5 from whence the air flows through air inlet nozzles 3a and 3b into UVC treatment chamber 1. Long sides 93 and 94 are dimensioned to fit standard suspended ceiling systems of 2 feet×4 feet, for example. This allows installation of the UVC air treatment system in accordance with this invention in office spaces, for example, that may not have extra room for a UVC air treatment system.

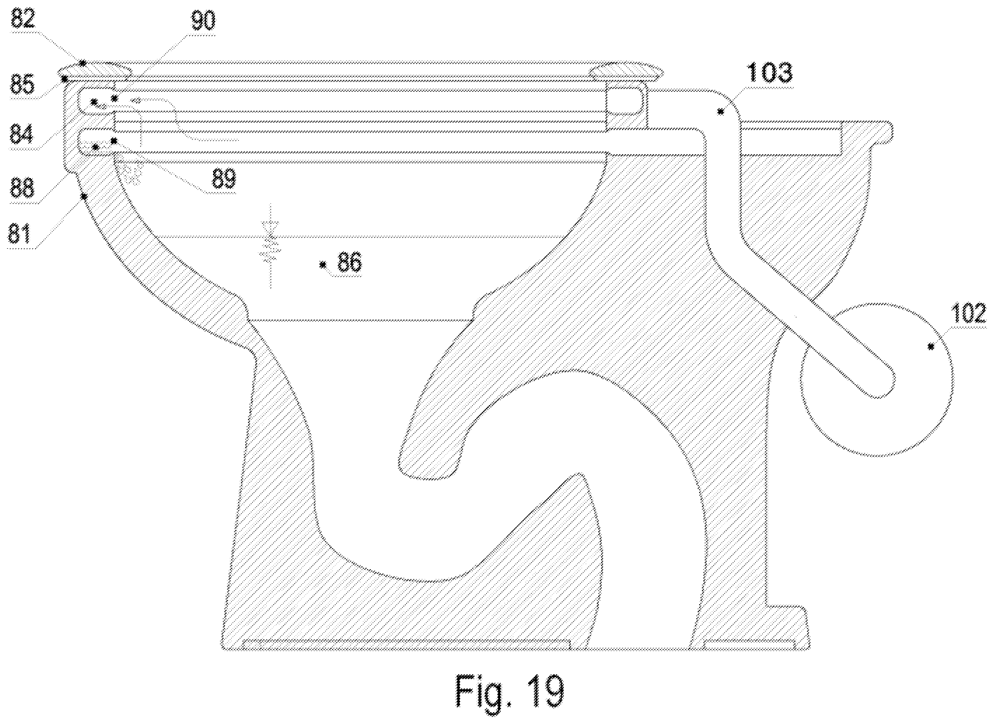

Referring to FIG. 19, a toilet vent arrangement for use with the UVC air sterilizer of the present invention is illustrated. Toilet seat 82 incorporates toilet seat spacers that rest on toilet bowl rim 85. Exhaust duct 84 is integral with the porcelain toilet bowl 81. Water conduit 88 supplies flush water to the toilet bowl 81 through ports 89 in the conventional manner. Water level 86 is shown for reference. Ports 90 admit foul air from toilet bowl 81 into exhaust duct 84 that carries it to the UVC air sterilizer and associated fans and filters.

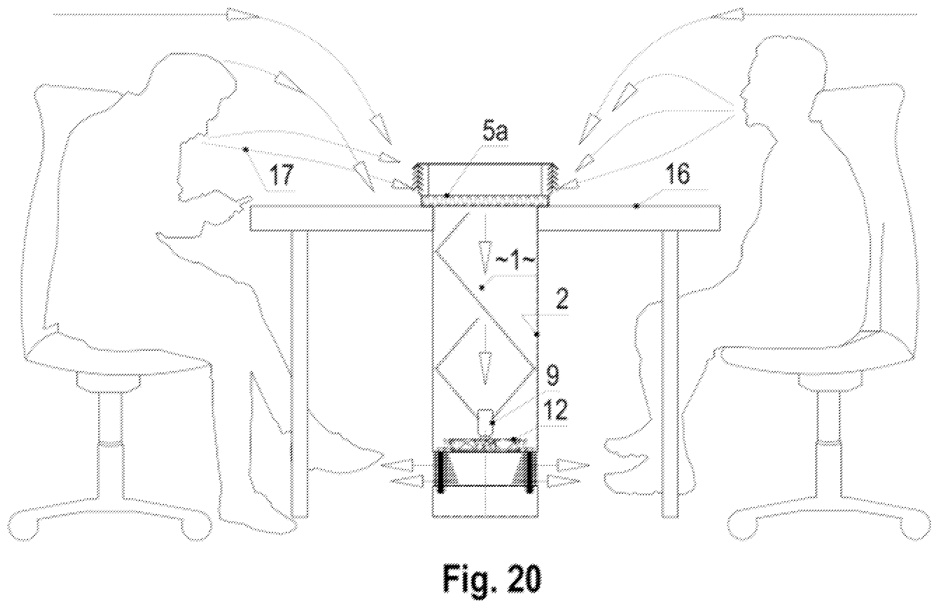

Referring to FIG. 20, a UVC air sterilizer is shown incorporated into a table or situated between tables for the purpose of vacuuming up any pathogens seated at table 16. The illustrated system includes filter 51, treatment chamber 1, UVC source 9 and blowers 12. The system would be sized so that the approach velocity of air to the UVC air sterilizer would entrain breathed air 17 from those seated at the table. This protects not only those seated at the table from rebreathing unsterilized air from others, but also protects all of the other occupants of the establishment, such as an office or restaurant, from infection from thos seated at any table so equipped. Filter 5a establishes laminar flow within treatment chamber 1. Reflective surfaces 2 line the treatment chamber in order to maximize the utilization of UVC radiation from lamp 9. Prior art systems provide uniformity of pathogen particle treatment too low for this application and, as a result of poor aerodynamic efficiency may generate intolerable noise if operated at the flowrate required for breath capture.

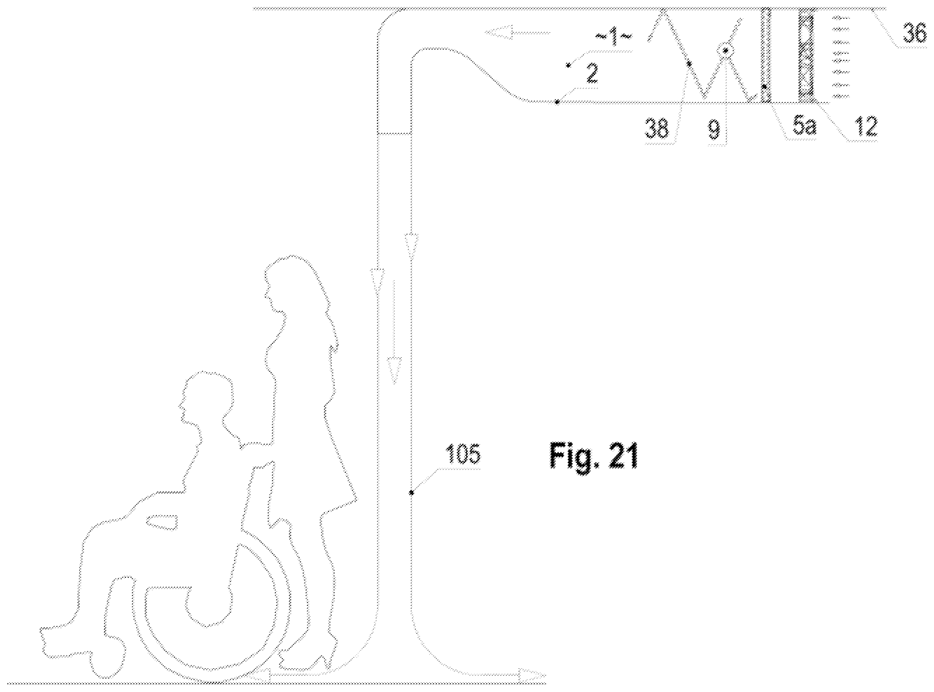

Referring to FIG. 21, a UVC air sterilizer in accordance with the present invention is shown in conjunction with an air curtain. This conveniently provides a barrier to pathogen spread in hospital corridors, shop entrances, aircraft, trains, and so forth. This application is enabled by the unique combination of high pathogen kill probability, low power consumption and low noise levels provided in accordance with the present invention.

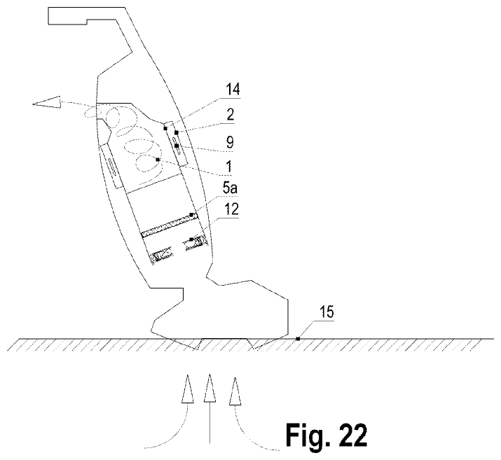

Referring to FIG. 22, A vacuum cleaner is illustrated that is comprised of a blower 12, a filter 5a, a UVC treatment chamber 1, a UVC transparent enclosure 14, a UVC reflective surface 2 and UVC lamps 9. The treatment chamber is designed for minimal turbulence and maximum UVC path length in accordance with the present invention.

It should be noted that the unique accelerating free vortex flow path that is a salient part of many of the embodiments of this invention suppresses turbulence and allows no parcels of air to take a short cut form UVC treatment chamber inlet to the UVC treatment chamber outlet. All the transiting air follows a uniform path length with a transit time that does not materially vary, while the high proportion of reflective surface provides a uniformly high UVC intensity within the treatment chamber. The UVC radiation is reflected so many times that after the first few reflections it has dispersed uniformly throughout the UVC treatment chamber.

It should be noted that proprietors of some of the prior art brag about the benefits of turbulence as a means of exposing all sides of a pathogen. In the case of the present invention the number of reflections of the UVC radiant energy is enough to render the UVC flux within the UVC treatment chamber isotropic. This isotropic radiant UVC flux is beneficial on account of not needing otherwise undesirable turbulence to achieve exposure on all sides of transiting pathogens and is also beneficial in providing six times the exposure provided by a single axis unidirectional UVC flux. In the case of the present invention, UVC flux is maintained at a uniformly high level in two directions along each of three axes. This isotropic flux is the result of the unique combination of low UVC loss at air inlets and outlets, materials of high diffuse reflectivity, a chamber configured for long path lengths, fewer required reflections, and an overall longer mean total path, from emission at the lamp to eventual absorption, for each UVC photon.

The nozzle configuration associated with the aforementioned embodiments functions very nearly isentropically, i.e., with negligible energy loss as air is accelerated through the nozzle into the UVC treatment chamber. Furthermore, almost no energy is lost as air transits the treatment chamber as it follows a free vortex spiral toward the central chamber exit. The exiting air has accumulated kinetic energy that may be largely recovered by either of several methods or by a combination thereof. In the case of an axial flow blower at the UVC treatment chamber exit, the velocity vectors of the exiting air may be precisely coordinated with bespoke blades to eliminate nearly all tangential velocity in the air exiting the fan. The axial velocity of air exiting the fan may then be substantially recovered by means of a conical diffuser or equal. Air being treated may thus be accelerated into the treatment chamber where the induced free vortex suppresses turbulence, and then decelerated again as it passes through the fan and subsequent diffuser. The inlet filter pressure drop can be controlled by selecting the size and quantity of filters and the number of nozzles to use in conjunction with the UVC treatment chamber under consideration. Finer filters with a lower flow rating may be compensated for by increasing the quantity and area of the filters.

As an alternative to swirling air entering the blower directly from the treatment chamber, a set of diffuser vanes may be provided that efficiently remove the swirl component from the exiting air. In this manner the air may enter a standard axial flow fan operating at its design point. It should be noted that such a standard axial flow fan will leave a residual tangential velocity component in the exiting flow. The kinetic energy represented by this tangential velocity component may not be entirely recoverable. This is not a disadvantage peculiar to this invention but is due rather to the nature of axial flow fans without guide vanes and applies to prior art air treatment systems incorporating axial flow fans without guide vanes.

The use of diffuser vanes at the UVC treatment chamber central outlet also allows the tangential kinetic energy of the rotating air to be recovered as the air exits the UVC treatment chamber without the use of a special fan for that purpose.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “means for treating air with UVC light” or a “UVC air treatment system” should be understood to encompass disclosure of the act of “treating air”—whether explicitly discussed or not—and, conversely, were there effectively disclosure of the act of “treating air”, such a disclosure should be understood to encompass disclosure of a “UVC air treatment system” and even a “means for treating air”. Such changes and alternative terms are to be understood to be explicitly included in the description. The light in the ultraviolet-C bandwidth is specified because it is the most efficient wavelength to accomplish the serialization of air as specified herein. However, light in the ultraviolet-A and ultraviolet-B bandwidths may also be used.

The term UVC is meant broadly to include, not only UVC radiation, but also UVA and UVB as alternatives that may be used in some instances.

Thus, the applicant(s) should be understood to claim at least: i) each of the input devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the elements disclosed, xi) each potentially dependent claim or concept as a dependency on each and every one of the independent claims or concepts presented, xii) processes performed with the aid of or on a computer as described throughout the above discussion, xiii) a programmable apparatus as described throughout the above discussion, xiv) a computer readable memory encoded with data to direct a computer comprising means or elements which function as described throughout the above discussion, xv) a computer configured as herein disclosed and described, xvi) individual or combined subroutines and programs as herein disclosed and described, xvii) the related methods disclosed and described, xviii) similar, equivalent, and even implicit variations of each of these systems and methods, xix) those alternative designs which accomplish each of the functions shown as are disclosed and described, xx) those alternative designs and methods which accomplish each of the functions shown as are implicit to accomplish that which is disclosed and described, xxi) each feature, component, and step shown as separate and independent inventions, and xxii) the various combinations and permutations of each of the above.

It should also be understood that for practical reasons and so as to avoid adding potentially hundreds of claims, the applicant may eventually present claims with initial dependencies only. Support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. Further, if or when used, the use of the transitional phrase “comprising” is and will be used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

Patents, publications, or other references mentioned in this application for patent are hereby incorporated by reference. In addition, as to each term used it should be understood that unless its utilization in this application is inconsistent with such interpretation, both traditional and common dictionary definitions should be understood as incorporated for each term and all definitions, alternative terms, and synonyms such as contained in the Random House Webster's Unabridged Dictionary, second edition are hereby incorporated by reference. Finally, all references listed in this application are hereby appended and hereby incorporated by reference, however, as to each of the above, to the extent that such information or statements incorporated by reference might be considered inconsistent with the patenting of this/these invention(s) such statements are expressly not to be considered as made by the applicant(s). Please be aware that cited works of non-patent literature such as scientific or technical documents or the like may be subject to copyright protection and/or any other protection of written works as appropriate based on applicable laws.

Copyrighted texts may not be copied or used in other electronic or printed publications or re-distributed without the express permission of the copyright holder.

Us Patents

| | Kind | | Name of Patentee or |
|---|---|---|---|
| Patent Number | Code | Issue Date | Applicantof cited Document |
| 9,914,133 | B2 | 2018 Mar. 13 | Lev |
| 9,005,347 | B2 | 2015 Apr. 14 | Lev |
| 8,841,640 | B1 | 2014 Sep. 23 | Abbott et al. |
| 7,828,882 | B2 | 2010 Nov. 9 | Dietz |
| 6,949,223 | B2 | 2005 Sep. 27 | McEllen |
| D829312 | S | 2018 Sep. 25 | Riering-Czekalla et al. |
| D838354 | S | 2019 Jan. 15 | Farone |
| D835764 | S | 2018 Dec. 11 | Farome et al |
| D812209 | S | 2018 Mar. 6 | Farome et al. |
| 5,124,131 | | 1992 Jun. 23 | Wekhof |
| 5,170,091 | | 1992 Dec. 8 | Wekhof |
| 5,144,146 | | 1992 Sep. 1 | Wekhof |
| 5,503,800 | | 1996 Apr. 2 | Free |
| 5,725,757 | | 1998 Mar. 10 | Binot |
| 5,832,361 | | 1998 Nov. 3 | Foret |
| 5,861,123 | | 1999 Jan. 19 | Schifftner |
| 9,127,206 | B2 | 2015 Sep. 8 | Foret |
| 63950723 | B1 | 2002 May 28 | Dauber |
| 9,855,362 | B1 | 2018 Jan. 2 | Engelhard |
| 10,039,852 | B2 | 2018 Aug. 7 | Yi et al. |
| 8,968,063 | B2 | 2015 Mar. 3 | Gifford et al. |
| 4,720,400 | | 1988 Jan. 19 | Manniso |

Us Patent Application Publications

| Publication | Kind | Publication | Name of Patentee or |
|---|---|---|---|
| Number | Code | Date | Applicantof cited Document |
| 20190031536 | A1 | 2019 Jan. 31 | Vardiel et al |
| 20130161251 | A1 | 2013 Jun. 27 | Foret |
| 20140110327 | A1 | 2014 Apr. 24 | Foret |
| 20140265004 | A1 | 2014 Sep. 18 | Foret |
| 20140299584 | A1 | 2014 Oct. 9 | Foret |
| 20170000145 | A1 | 2014 Jan. 5 | Foret |
| 20170001886 | A1 | 2017 Jan. 5 | Foret |
| 20110250099 | A1 | 2011 Oct. 13 | Bagwell et al. |
| 20050249630 | A1 | 2005 Nov. 10 | Odumuye et al. |
| 20050092932 | A1 | 2005 May 5 | Bircher et al. |
| 20050173351 | A1 | 2005 Aug. 11 | Neofotistos |
| 20170037307 | A1 | 2017 Feb. 9 | Foret |
| 20030217641 | A1 | 2003 Nov. 27 | Palestro et al. |
| 20130224086 | A1 | 2013 Aug. 29 | Stibich et al. |
| 20190060505 | A1 | 2019 Jan. 28 | Jaworski et al. |
| 20070266855 | A1 | 2007 Nov. 22 | Fleisher |
| 20190126202 | A1 | 2019 May 2 | Rao et al. |
| 20170354976 | A1 | 2017 Dec. 14 | Loreth |
| 20070036696 | A1 | 2007 Feb. 15 | McEllen et al. |
| 20040012583 | A1 | 2004 Jan. 22 | Burkdhardt |
| 20070207066 | A1 | 2007 Sep. 6 | Thur et al. |
| 20050061656 | A1 | 2005 Mar. 24 | Benoit et al. |
| 20080112854 | A1 | 2008 May 15 | Dunn et al. |
| 20030194599 | A1 | 2003 Oct. 16 | Sassa et al. |

Foreign Patent Documents

| Foreign | | | | Name of Patentee |
|---|---|---|---|---|
| Document | Country | Kind | Publica- | orApplicant of |
| Number | Code | Code | tionDate | cited Document |
| 20561778 | CN | U | 2016 May 25 | MEIZHOU RUITE |
| | | | | ELECTRICAL |
| | | | | APPLIANCE |
| | | | | TECH COLTD |
| 2019079281 | WO | A1 | 2019 Apr. 25 | Molekule Inc. |
| 0020045 | WO | | 2000 Apr. 13 | Common Services Agency |
| | | | | (GB/GB) |
| 104907175 | CN | A | 2015 Sep. 16 | HUANG JINGYUE |
| 10-2019- | KR | | 2019 Mar. 4 | ULSAN NAT INST |
| 0020442 | | | | SCIENCE & TECH UNIST |
| 209042633 | CN | U | 2019 Jun. 28 | SHENZHEN QUANXING |
| | | | | TECH CO LTD |
| 2009064368 | WO | A1 | 2009 May 22 | Gore Enterprise Holdings, |
| | | | | Inc. |
| 2005047381 | WO | A1 | 2005 May 26 | Gore Enterprise Holdings, |
| | | | | Inc. |
| 2004079208 | WO | A2 | 2004 Sep. 16 | Gore Enterprise Holdings, |
| | | | | Inc. |

Non-Patent Literature Documents

AirPurifiersandCleaners.com Duct UV Air Cleaner Systems, Brochure printed Apr. 3, 2020
Breath EZ UVC Light Technology to Improve indoor Air, Brochure printed Apr. 3, 2020
Oransi, How UV Light is Used i Air Purification, brochure printed Apr. 3, 2020
OION Technologies Operating Manual, Permanent Filter Iconic Air Purifier, Model No, B-1000,
Printed Apr. 3, 2020
Wek-tek, UV-Vortex-1L for R&D for UV-C Sanitation for Powders ad Shreds, Brochure
Copyright 2019
Wek-tek UVC-Production Tunnel with continuous high output amalgam UVC lamps, Brochure
copyright 2018
Wek-tek PUV-Pilot Tunnel with intense Pulsed UV Lamps, Brochure copyrigt 2018
Wek-tek Bench-top PEF R&D Sterilization System SBS-PEF-SA-1A, Brochure copyright 2016
Wek-tek PEF Sterilization System WT-PEF-PV1, Brochure copyright 2018
Wek-tek Intense Pulsed Light System XeMaticA-1L-Basic, Brochure copyright 2018
Wek-tek Intense Pulsed Light System XeMaticA-2L manually operated 1-2 lamps R&D System,
Brochure copyright 2018
Wek-tek Intense Pulsed Light System XeMaticA-2L-RepRate-V2 automatic R&D system with
two flash lamps, Brochure copyright 2020
GORE ® Diffuse Reflector, JK091020-01 Rev. Oct. 15, 2010
GORE ® Diffuse Reflector, Maximize light efficiency with longer product life, 1K080915 01 Rev.
Oct. 1, 2010
ePTFE Membrane, Device & Enclosure Protection with Unique Tetratex ® Technology, ePTFE
Membrane Device & Enclosure Protection with Unique Tetratex ® Technology
Phillips Scientific, Inc., ePTFE for INDUSTRIAL & MEDICAL Applications, Copyright 2020
Aeos ™ ePTFE Brochure, downloaded Apr. 18, 2020
Portex © Virtek ™ Fact Sheet: Sintered PTFE vs ePTFE - with differing structures come different
features and benefits, copyright 2018
3M High Strength Double Coated Tape with Adhesive 300 LSE, Technical Data, September 2016
Quill, T.; Weiss, S.; Hirschler C.; Pankadzh, V.; DiBattista, G.; Arthur, M.; and Chen J.;
Ultraviolet Reflectance of Microporous PTFE, Porex Corporation, 2016
Portex ® Virtek ™ PTFE Materials, Copyright 2018
Portex ® Virtek ™ PTFE Eletronics Materials Copyright 2015
Portex ® Reflective Media, printed May 6, 2020
Senwen Yang, Ph. D. Candidate, Cheng Zhang, M.A.Sc. Candidate, Chang Shu, Ph. D. Candidate,
Smart Air Curtains, Building and Civil Engineering, Concordia University
Moltei, M., Wired Magazine, the 60-year-old Scientific Screwup that Helped Covid Kill,
May 13, 2021

I claim:

1. An air purifier apparatus comprising:
(a) a UVC treatment chamber having an air inlet and an air outlet port
(b) an ultraviolet (UVC) light source within said chamber;
(c) a fan for moving ambient air through said air inlet, through said chamber, and outwardly through said air outlet;
wherein said chamber includes reflective interior surfaces for reflecting UV light along multiple pathways within said chamber to sterilize air moving therethrough between said air inlet and said air outlet; wherein said air inlet and said air outlet are adapted to prevent UVC light from escaping from said chamber.

2. Apparatus in accordance with claim 1, wherein said air inlet comprises a perforated panel adapted to reflect UV light back into said chamber.

3. Apparatus in accordance with claim 2, wherein said perforated panel comprises PTFE.

4. Apparatus in accordance with claim 1, wherein said air outlet comprises an exhaust plenum adapted to inhibit escape of UV light from said chamber.

5. Apparatus in accordance with claim 1, further comprising a diffuser connected to said air outlet.

6. Apparatus in accordance with claim 1, wherein said air inlet nozzle is oriented at a right angle to the UV light source.

7. Apparatus in accordance with claim 1, further comprising curved baffle within said chamber adjacent said air inlet for imparting spiral flow to air entering into said chamber.

8. Apparatus in accordance with claim 1, wherein there are multiple air inlet nozzles.

9. Apparatus in accordance with claim 1, wherein said chamber comprises multiple curved interior surfaces.

10. Apparatus in accordance with claim 1, further comprising a photocatalyst panel positioned between said air inlet and said UV light source.

11. Apparatus in accordance with claim 1, wherein said air inlet includes a filter.

12. Apparatus in accordance with claim 2, wherein said perforated panel comprises a plurality of air inlet nozzles.

13. Apparatus in accordance with claim 1, wherein said air inlet comprises a movable nozzle which is adapted to reduce the size of an air inlet opening when air flow rate through said air inlet becomes reduced.

14. Apparatus in accordance with claim 1, wherein said air outlet comprises a tubular pipe supporting said chamber; wherein said tubular member includes a base having at least one opening for treated air to exit the tubular member near the floor.

15. Apparatus in accordance with claim 1, wherein said chamber mounted to the ceiling of a room.

16. Apparatus in accordance with claim 1, wherein said chamber is configured to provide a free vortex air flow path from said air inlet to said air outlet.

17-21. (canceled)

22. A method for purifying air comprising the steps of:
(a) providing a chamber having an air inlet and an air outlet;
(b) providing an ultraviolet (UV) light source within said chamber;
(c) providing a fan for moving ambient air through said air inlet, through said chamber and through said air outlet;
wherein said chamber includes reflective interior surfaces for reflecting UV light along multiple pathways within said chamber to sterilize air moving therethrough between said air inlet and said air outlet; wherein said air inlet and said air outlet are adapted to prevent UV light from escaping from said chamber.

23. A method in accordance with claim 22, wherein said air inlet comprises a perforated panel adapted to reflect UV light back into said chamber.

24. A method in accordance with claim 23, wherein said perforated panel comprises PTFE.

25. A method in accordance with claim 22, wherein said air inlet is oriented at a right angle to said UV light source.

26. A method in accordance with claim 22, wherein said chamber further comprises curved baffle means adjacent said air inlet for imparting spiral flow to air entering said chamber.

27-32. (canceled)
